(19) United States
(12) Patent Application Publication
(10) Pub. No.: US 2017/0058343 A1
(43) Pub. Date: Mar. 2, 2017

(54) SYSTEMS AND METHODS FOR COLOR DETECTION IN HIGH-THROUGHPUT NUCLEIC ACID SEQUENCING SYSTEMS

(71) Applicant: QIAGEN Instruments AG, Hombrechtikon (CH)

(72) Inventors: Harald Quintel, Hombrechtikon (CH); Konstantin Lutze, Hombrechtikon (CH)

(21) Appl. No.: 15/253,015

(22) Filed: Aug. 31, 2016

Related U.S. Application Data
(60) Provisional application No. 62/212,820, filed on Sep. 1, 2015.

(51) Int. Cl. C12Q 1/68; H04N 5/33; G02B 5/20; G02B 27/14; G02B 26/08; H04N 9/04; H04N 5/225

(52) U.S. Cl. CPC C12Q 1/6874; H04N 9/045; H04N 5/332; H04N 5/2254; G02B 27/141; G02B 26/0883; H04N 5/2256; G02B 26/0875; G02B 5/201

(57) ABSTRACT

A sequencing instrument optical system having a combined light source with multiple collinear excitation beams having different respective excitation wavelengths, a sequencing surface having DNA templates and nucleobase labels configured to emit a respective emission light at a different respective emission wavelength upon excitation by one or more of the excitation beams, a color camera configured to detect the emission light of each of the nucleobase labels, a first optical pathway configured to direct the collinear excitation beams from the combined light source to the sequencing surface, and a second optical pathway configured to direct the emission light from the sequencing surface to the color camera.

### CROSS REFERENCE TO RELATED APPLICATIONS

This application claims priority to U.S. Provisional Application No. 62/212,820, entitled SYSTEMS AND METHODS FOR COLOR DETECTION IN HIGH-THROUGHPUT NUCLEIC ACID SEQUENCING SYSTEMS, filed Sep. 1, 2015, the contents of which is incorporated fully herein by reference.

### BACKGROUND OF THE INVENTION

Field of the Invention

The present invention relates generally to instruments for detecting fluorescing dyes or other light-emitting labels associated with nucleobases used during sequencing-by-syntheses or other sequencing processes.

Description of the Related Art

DNA sequencing processes are used to determine the order of base pairs within a DNA molecule. This technology has a variety of uses, such as determining the identity of a DNA molecule or whether the DNA molecule includes particular features (e.g., features indicative of congenital conditions), and so on. A number of technologies are available to determine DNA sequences. For example, in a typical sequencing by synthesis (SBS) process, specially-designed nucleotides and DNA polymerases may be used to read the sequence of surface-bound, single-stranded DNA templates in a controlled manner. This process uses labels (also known as probes or tags) to identify the particular nucleobases (adenine, guanine, cytosine and thymine) that make up the DNA molecule. Other sequencing technologies may use native nucleotides and/or polymerases or labeled oligonucleotides and ligation enzymes to determine nucleic acid sequences.

In its most basic sense, the SBS process operates by extending the length of a DNA template molecule one nucleobase at a time, and recording the sequence of added nucleobases. More specifically, the process extends the DNA template by one nucleobase, and optically examines (“reads”) the resulting molecule to determine whether (or what kind of) a label is present at the DNA template location. The presence of a label indicates that a nucleobase associated with that particular label has been added to the DNA template. This process is then repeated multiple times to determine a sequence of base pairs that make up the DNA templates. To increase the processing speed and make this process more practical, it typically is desirable to process many millions of DNA templates, each of which may comprise a fragment of a larger DNA molecule. For example, millions of DNA templates may be placed in ordered or random locations (“template spots”) on a sequencing surface, and processed together. Each DNA template may itself comprise a single molecule or multiple essentially identical molecules. After the DNA templates are processed to determine their sequences, the individual sequences may be compared to one another and collated to identify the nature of the original complete DNA molecule.

Conventional SBS methods and other methods that rely on optically examining nucleobase labels must operate at the very small scale of the DNA templates and the low illumination intensity of the nucleobase labels. A typical nucleobase label comprises a fluorescent molecule having a fluorophoric compound that emits light when it is excited by an external “excitation” light source. The wavelength of the emitted light depends on the particular fluorophore. The mean wavelength of the emitted light generally is slightly greater than the mean wavelength of the excitation light due to a loss of energy of the photons (a phenomenon known as the Stokes shift). The intensity of the light is very low, which can be addressed to some degree by amplifying each DNA template in situ to aggregate multiple identical nucleobase labels at each template spot. However, even with such amplification it is necessary to take measures to carefully distinguish the signal generated by the nucleotide label from background noise and from other labels that may be nearby.

In some cases, the process of extending and reading is performed serially by presenting only a single kind of nucleobase (e.g., adenine) to join the DNA templates in each extension step, performing a read to detect which of the DNA templates have been extended, and then repeating the same process individually for each of the remaining nucleobases (guanine, cytosine and thymine). This serial process minimizes the possibility that one nucleobase will be mistaken for another during the read step, because only one kind of nucleobase can be added during each extension and read cycle. However, this process is time consuming because it requires a large number of processing steps: four complete extension cycles and four complete read cycles to extend all of the DNA templates by a single base pair.

In other cases, the extension step can be performed in parallel by presenting some or all of the nucleobases to the DNA templates during each extension cycle. This method speeds up the process, because each DNA template theoretically will be extended during each extension cycle. However, this process may still require four different reading steps in series to accurately identify the labels associated with each type of nucleobase. This process also may require more demanding optical performance than a serial process, because some nucleobase labels have similar illumination wavelengths (such as green and yellow, or red and dark red), which may make differentiation between these labels more difficult.

Typical SBS instruments are configured to read each of the four types of nucleobase label during multiple separate process steps. Such devices may employ moving optics, such as shown in the prior art example in FIG. 1. In this example, the instrument 100 includes a sequencing surface 102 on which the DNA templates are deposited. The sequencing surface 102 may comprise any suitable surface on which DNA templates are located, such as a chip, a bead, a flow cell or other structure through which reagents are passed to perform the various chemistry steps necessary to extend the DNA templates, or a combination thereof. The sequencing surface 102 is examined by a fixed CCD (charge-coupled device) camera 104 by way of an objective lens 106. The CCD camera sensor typically does not include local color filters so that it only determines the light intensity and not the wavelength. This has been preferred because light filters reduce the emitted light intensity, and typically decrease the spatial resolution of the sensor. Thus, the camera 104 itself is unable to differentiate between the different nucleobase labels, and the instrument 100 must read each of the four nucleobase labels during a separate respective read operation. To do this, the instrument 100 includes four separate illumination modules 108, each having a light source 110 (e.g., LED lights or the like), an excitation filter 112, a dichroic mirror 114 and an emission filter 116. Various light guides 120 and the like also may be used throughout the system to shield the light path between optical components. Line A illustrates the travel path of the light from the light source 110 to the camera 104.

The illumination modules 108 typically are configured to maximize the intensity of the emitted light for each particular nucleobase label. For example, if a particular nucleobase label absorbs excitation light having a wavelength of 495 nanometers (“nm”) and emits light at a wavelength of 520 nm, the light source 110 may be selected to emit high intensity light at a wavelength of around 495 nm, the excitation filter 112 may be selected to filter the excitation light to a narrow band surrounding 495 nm, the dichroic mirror 114 may be selected to reflect light at around 495 nm and transmit light at around 520 nm, and the emission filter 116 may be selected to filter the emitted light to a narrow band surrounding 520 nm. The use of such filters and a dichroic mirror can help prevent the light source 108 from inadvertently exciting other nucleobase labels and providing false reads, or otherwise saturating or affecting the operation of the camera 104.

After the extending step, the sequencing surface 102 is read in four steps. Between each reading step, the instrument 100 mechanically moves a different illumination module 108 to position the new module's dichroic mirror 114 and emission filter 116 between the objective lens 106 and the camera 104. The optics used to detect each individual label must be accurately and repeatably aligned in order to accurately compare reads at individual DNA template spots during subsequent extensions and/or reads, because even a very minor misalignment may make it impossible to correlate the locations of the DNA templates from one read to the next. Such optics typically are expensive to make and may require stringent and frequent calibration and service.

Some instruments also employ a movable sequencing surface stage 118 and/or moving objective lens 106. Such motility may be desirable, for example, to examine a sequencing surface 102 that is larger than the field of view of the camera 104, to allow the sequencing surface 102 to be removed from the optical system during other processing steps, or to move the sequencing surface 102 into proper registration with the image sensor. In such devices, the demand increases to have highly accurate and repeatable alignment between the various optical components. At the magnification required to examine and differentiate individual DNA templates, a small misalignment of the surface can cause a dramatic shift in the field of view of the optical system. Thus, systems that do not have a fixed sequencing surface 102 may require sophisticated software techniques computationally align the data from each read step to provide a correct base pair sequence for each individual DNA template.

Examples of devices and similar technology are shown in U.S. Patent Application Publication Nos.: 2014/0267669 and 2009/0298131, and U.S. Pat. Nos. 8,940,481 and 8,481,259, all of which are incorporated herein by reference.

While the prior art provides certain useful instruments and advances, the present inventors have determine that there continues to be a need to advance the state of the art of sequencing instruments.

### SUMMARY

In one exemplary embodiment, there is provided a sequencing instrument optical system having a combined light source with a number of collinear excitation beams, each excitation beam having a different respective excitation wavelength, a sequencing surface having a number of DNA templates and a number of nucleobase labels configured to emit a respective emission light at a different respective emission wavelength upon excitation by one or more of the excitation beams, a color camera configured to detect the emission light of each of the nucleobase labels, a first optical pathway configured to direct the collinear excitation beams from the combined light source to the sequencing surface, and a second optical pathway configured to direct the emission light from the sequencing surface to the color camera.

In the first exemplary embodiment, the combined light source may have four collinear excitation beams, and the combined light source may have a first light source and at least one additional light source directed onto a collinear path with the first light source by a dichroic mirror.

In the first exemplary embodiment, the color camera may have a sensor having a number of photosensitive pixels, and a filter array having a number of color filters, each color filter being associated with a respective photosensitive pixel. The color filters may include red color filters, green color filters, and blue color filters.

The filter array of the first exemplary embodiment may be a hyperspectral filter. In this embodiment, the color filters may be a number of Fabry-Perot spectral filters. The color filters may include a first group of filters configured to transmit light having a first wavelength associated with a first nucleobase label emission light, a second group of filters configured to transmit light having a second wavelength associated with a second nucleobase label emission light, a third group of filters configured to transmit light having a third wavelength associated with a third nucleobase label emission light, and a fourth group of filters configured to transmit light having a fourth wavelength associated with a fourth nucleobase label emission light. The first, second, third and fourth wavelengths associated with the first, second, third, and fourth nucleobase label emission lights may each include a wavelength corresponding to a respective first, second, third and fourth peak emission wavelength of the respective nucleobase label. The first peak emission wavelength may be about 525 nm, the second peak emission wavelength may be about 565 nm, the third peak emission wavelength may be about 630 nm, and the fourth peak emission wavelength may be about 680 nm. The first, second, third and fourth wavelengths may also include a respective range of wavelengths surrounding the respective peak emission wavelength. In some examples, the respective ranges of wavelengths may not exceed a range of 20 nm, or a range of 5 nm. The ranges of wavelengths may not include any overlapping wavelengths.

In one embodiment, the filter array may include a first group of filters, a second group of filters, a third group of filters, and a fourth group of filters that are arranged in a mosaic pattern. In another embodiment, the groups of filters may be arranged in a scanning pattern with each group of filters arranged in a continuous row.

The sequencing surface may be movable in a first direction relative to the color camera, and the first optical path may include a lens assembly configured to project the collinear excitation beams onto the sequencing surface in a line perpendicular to the first direction.

The color camera may be a multi-sensor camera having a number of sensors. There may be three or four sensors configured to receive emission light having a different wavelength. The color camera also may be a hyperspectral camera and the number of sensors includes a first sensor configured to detect a first emission wavelength, a second sensor configured to detect a second emission wavelength, a third sensor configured to detect a third emission wavelength, and a fourth sensor configured to detect a fourth emission wavelength. The first, second, third and fourth emission wavelengths may include respective first, second, third and fourth peak emission wavelengths of respective first, second, third and fourth nucleobase labels. The first, second, third and fourth emission wavelengths also may each include a range of wavelengths not exceeding 20 nm, or not exceeding 5 nm. The first, second, third, and fourth emission wavelengths also may not include any overlapping wavelengths. A multi-sensor color camera may include a number of prisms, each of which is configured to direct a respective emission light to a respective sensor.

The first optical pathway and the second optical pathway may include a shared multiband dichroic mirror configured to transmit the emission light, and reflect the number of collinear excitation beams towards the sequencing surface. At least one of the first optical pathway and the second optical pathway may be oblique to the sequencing surface.

In another exemplary embodiment, there is provided a sequencing instrument optical system having a first excitation beam having a first excitation wavelength, a second excitation beam having a second excitation wavelength that is different from the first excitation wavelength, and a sequencing surface. The sequencing surface has a number of DNA templates, a first nucleobase label configured to emit a first emission light at a first emission wavelength upon excitation by the first excitation beam, and a second nucleobase label configured to emit a second emission light at a second emission wavelength upon excitation by the second excitation beam. The instrument also includes a first lens assembly configured to project the first excitation beam onto a first location on the sequencing surface in a line perpendicular to the first direction, a second lens assembly configured to project the second excitation beam onto a second location on the sequencing surface in a line perpendicular to the first direction, the second location being different from the first location, and a sensor configured to detect the emission light of each of the nucleobase labels and configured to be movable in a first direction relative to the sequencing surface. A first color filter configured to transmit the first emission wavelength is located between the first location on the sequencing surface and a first part of the sensor, and a second color filter configured to transmit the second emission wavelength is located between the second location on the sequencing surface and a second part of the sensor.

The second exemplary embodiment also may include a third excitation beam having a third excitation wavelength, a third nucleobase label configured to emit a third emission light at a third emission wavelength upon excitation by the third excitation beam, a third lens assembly configured to project the third excitation beam onto a third location on the sequencing surface in a line perpendicular to the first direction, the third location being different from the first location and the second location, and a third color filter configured to transmit the third emission wavelength and located between the third location on the sequencing surface and a third part of the sensor. The embodiment also may include a fourth excitation beam having a fourth excitation wavelength, a fourth nucleobase label configured to emit a fourth emission light at a fourth emission wavelength upon excitation by the fourth excitation beam, a fourth lens assembly configured to project the fourth excitation beam onto a fourth location on the sequencing surface in a line perpendicular to the first direction, the fourth location being different from the first location, the second location and the third location, and a fourth color filter configured to transmit the fourth emission wavelength and located between the fourth location on the sequencing surface and a fourth part of the sensor.

In the second exemplary embodiment, one or more lenses may be provided to project the first emission wavelength along a first discrete line at the first part of the sensor, and to project the second emission wavelength along a second discrete line at the second part of the sensor.

In the first or second exemplary embodiment, the sequencing surface may be mounted on a movable stage to thereby make the sensor movable in a first direction relative to the sequencing surface.

Other alternatives will be apparent to persons of ordinary skill in the art in view of the present disclosure.

The recitation of this summary of the invention is not intended to limit the claims of this or any related or unrelated application. Other aspects, embodiments, modifications to and features of the claimed invention will be apparent to persons of ordinary skill in view of the disclosures herein.

### BRIEF DESCRIPTION OF THE DRAWINGS

A better understanding of the exemplary embodiments may be understood by reference to the attached drawings, in which like reference numbers designate like parts. The drawings are exemplary and not intended to limit the claims in any way.

FIG. 1 is schematic diagram of a prior art sequencing instrument optical system.

FIG. 2 is a schematic diagram of a first embodiment of an instrument optical system.

FIG. 3 is a schematic diagram of a portion of a conventional color digital image sensor.

FIG. 4 is a schematic diagram of a portion of a first hyperspectral digital image sensor.

FIG. 5 is a schematic diagram of a Fabry-Perot spectral filter.

FIG. 6 is a schematic diagram of a second embodiment of a sequencing instrument optical system.

FIG. 7 is a schematic diagram of a third embodiment of a sequencing instrument optical system.

FIG. 8 is a schematic diagram of a fourth embodiment of a sequencing instrument optical system.

FIG. 9 is a schematic diagram of a fifth embodiment of a sequencing instrument optical system.

### DETAILED DESCRIPTION

It has been determined that SBS instruments and other instruments that optically read labeled nucleobases or other chemical labels may be beneficially modified in various ways, and particularly by reducing or eliminating the need to mechanically move the instrument's optical components between successive nucleobase label reads. This description provides several examples of instrument optical systems that may provide one or more benefits as compared to existing systems, such as increased speed, greater reliability, greater accuracy, lower cost, or the like.

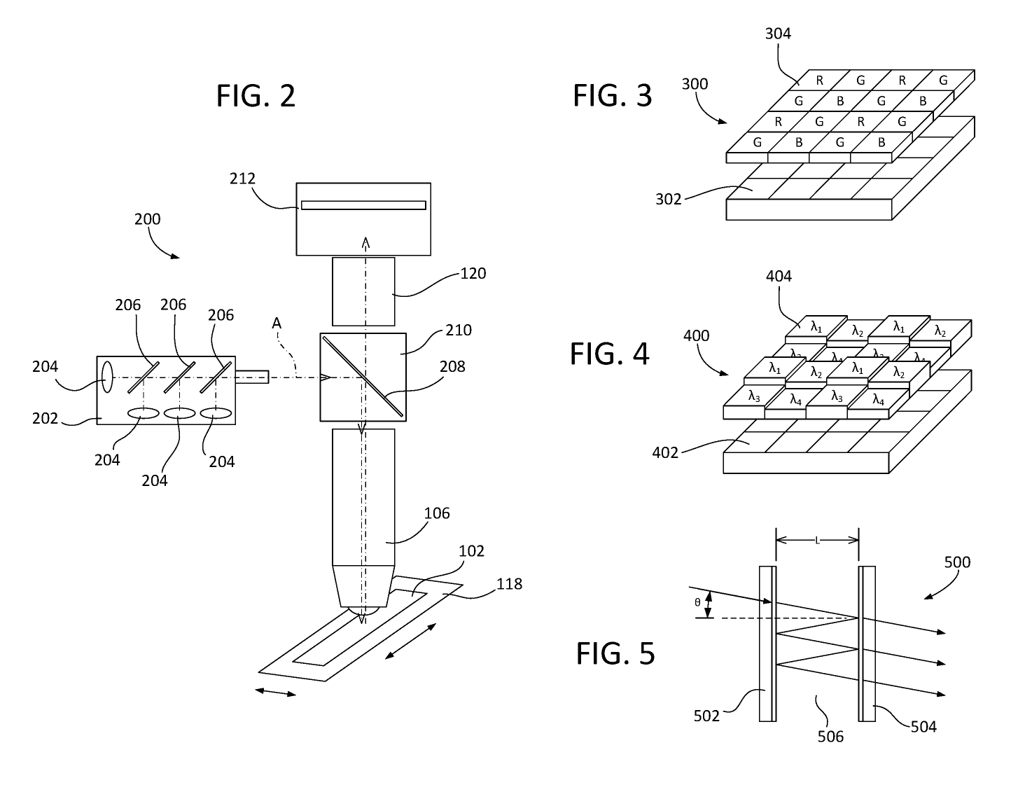

A first exemplary embodiment of an optical system for a sequencing instrument 200 is schematically illustrated in FIG. 2. Instrument 200 uses a combined light source 202 that generates light at one or more wavelengths selected to excite two, three, or all four of the nucleobase labels that are to be used during the sequencing process. In the shown example, the combined light source 202 includes first, second, third and fourth light sources 204. Each light source 204 is selected to emit light at an excitation wavelength selected to excite one of the nucleobase labels, such as blue (e.g., 470 nm), green (e.g., 520 nm), yellow (e.g., 570 nm) and red (e.g., 615 nm). The nucleobase labels may include fluorescing dyes (e.g., Alexa 488, Cy3, Texas Red and Cy5) or other compositions associated with particular nucleotides, such as described in U.S. Pat. No. 8,481,259, which is incorporated herein by reference. Other compositions, labels and fluorophores known in the art or later developed, having different excitation and emission wavelengths, may be used in these or other embodiments.

Fluorophores used in nucleobase labels oftentimes can be excited by a range of different incoming wavelengths. As such, a light source selected to excite one kind of nucleobase label also might excite other nucleobase labels to some degree. In some cases, a single source might be used to effectively excite two or more labels. However, it is more preferred to have a single light source operated at or near the most efficient wavelength to excite each individual nucleobase label. Examples of suitable light sources 204 include lasers, LED lights, diodes, and other light sources that are configured or filtered to emit the desired wavelength. Such devices are known in the art and need not be described in detail herein.

The light sources 204 are configured to emit beams that are collinear (i.e., aligned along the same straight line) along a single axis, as shown by arrow A. This may be accomplished by directing one light source 204 along the desired axis, and using mirrors 206 to redirect the remaining light sources 204 along the same axis. The mirrors 206 may comprise dichroic mirrors or the like, which allow the wavelength(s) of the upstream light source(s) to pass through the back surface, but reflect the wavelength of the particular light source 206 that is being redirected. The beams alternatively may be directed along a common axis by passing them through one or more prisms or by other methods and devices, as known in the art.

Each light source 204 preferably is configured to generate light having a single wavelength, or a very narrow range of wavelengths (e.g., light within a range of about 20-30 nm). As used herein, a “range of wavelengths” refers to a continuous portion of the spectral range spanning a difference of wavelength values. For example, a range of wavelengths not exceeding 20 nm may include a 20 nm-wide portion of the electromagnetic spectrum (e.g., from 520 nm to 540 nm) as measured at the full-width at half maximum value of the combined intensity profile of the wavelengths. Using this measurement technique, the light still may include wavelengths outside the defined range, but in relatively small amounts. This may be accomplished by using light sources that naturally emit only a narrow range of wavelengths (e.g. laser diodes), or by using additional optical elements to filter out undesired wavelengths. For example, a bandpass filter may be positioned between a light source 204 and its associated mirror 206, or a mirror 206 may comprise a dichroic mirror that only reflects a narrow range of desired wavelengths. Optical filters, dichroic mirrors, and the like are available from a variety of sources, such as Edmund Optics Inc. of Barrington, N.J.

The collinear combined beam A is reflected off a mirror 208, which redirects the beam through the objective lens 106 and to the sequencing surface 102. The sequencing surface 102, which may be a chip, bead, flow cell, or other suitable substrate or combination of substrate types, includes a plurality of DNA templates to which nucleobase labels have been attached through a prior extension step, but it is also contemplated that embodiments may be readily used for observing the sequencing process during the extension step. The sequencing surface 102 optionally may comprise a flat planar surface that extends orthogonally from the axis of the collinear combined beam A at the point at which the beam A impinges upon the sequencing surface 102. Each nucleobase label may be excited by at least one of the excitation wavelengths provided by the collinear combined beam A. The collinear combined beam A simultaneously excites all of the nucleobase labels that are sensitive to the incoming beam wavelengths, which causes the nucleobase labels to fluoresce at their respective emission wavelengths. The emitted light passes back through the objective lens 106, through the mirror 208, and to the camera 212. The mirror 208 preferably reflects the collinear combined excitation beams, but transmits the emitted light from the nucleobase labels. To this end, the mirror preferably comprises a multiband dichroic mirror having transmission wavelengths matching each of the nucleobase label emission wavelengths. Multiband and quad-band dichroic mirrors are available from Iridian Spectral Technologies of Ottawa, Ontario, Semrock, Inc. of Buffalo, N.Y., and other sources. One or more excitation filters (not shown) also may be provided in the optical path between the combined light source 202 and the mirror 208 to remove excitation light at wavelengths outside the desired ranges.

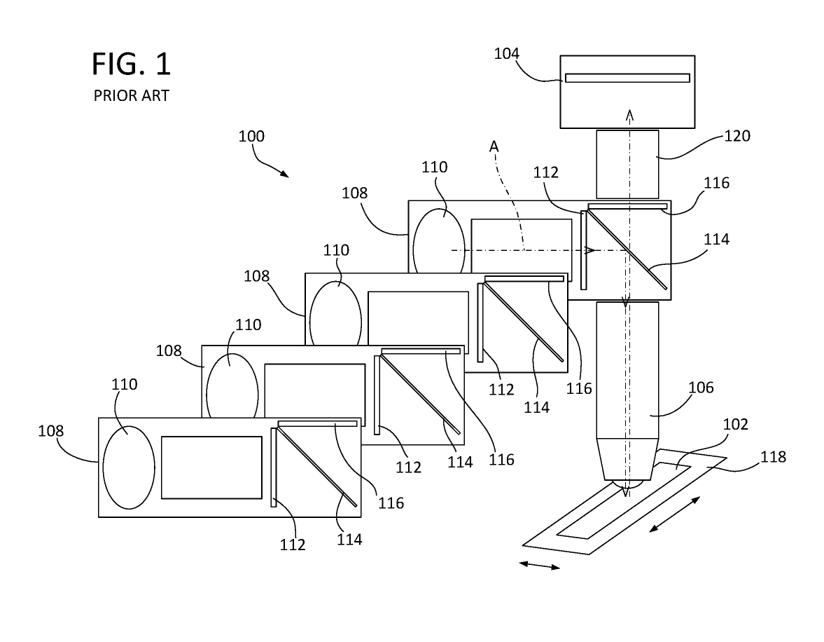

One or more emission filters (see FIG. 1) may be provided in the optical path between the mirror 208 and the camera 212. A typical nucleobase label emits light across a broad spectrum of wavelengths, but the majority of the light typically is emitted at a particular wavelength or narrow band of wavelengths (the “emission wavelength”). An emission filter may be used to narrow the range of emitted light to the emission wavelength or a small range (e.g., 20-30 nm) surrounding the emission wavelength. This may be particularly helpful to limit light transmitted to the camera 212 to only the peak emission value for each of the four nucleobase labels to reduce ambiguity that might arise from reading the intensity of wavelengths that are produced by multiple different nucleobase labels.

It is also envisioned that a single multiband dichroic mirror that passes all four wavelengths may not be used in all embodiments. In such embodiments, multiple different mirrors may be provided as movable units 210, and mechanically moved into place to read the nucleobase labels during successive read operations. For example, one alternative embodiment may use four mirror units 210, each of which transmits a single emission wavelength. Another alternative embodiment may use two mirror units 210, each of which transmits two of the emission wavelengths. Where multiple mirrors are used, the read process will operate in a serial manner. Nevertheless, it is expected that limiting the moving parts to only the mirrors can still obtain cost, efficiency, and accuracy benefits. Other alternatives will be readily apparent to the person of ordinary skill in the art in view of this disclosure.

The camera 212 in this example may comprise a color camera that can simultaneously detect and differentiate between all of the emission wavelengths of the nucleobase labels used in the instrument (e.g., about 525 nm, about 565 nm, about 630 nm, and about 680 nm). This allows the reading process to be performed in one step when a single dichroic mirror 208 is used. Conventional color CCD and CMOS (complementary metal oxide semiconductor) sensors may be used for this purpose. Conventional color digital cameras use a color filter array located immediately over an array of photosites that detect the incoming photons. The color filter array includes filters in the red spectrum, green spectrum and blue spectrum. In typically color camera sensors, the filters are configures such that about twice as much green light is permitted to reach the sensor as compared to the other colors, so that the sensor image more accurately reflects the distribution of light sensitivity of the human eye.

FIG. 3 is a simplified schematic diagram of a portion of an exemplary conventional color CCD sensor 300. The sensor 300 includes a layer 302 of photosensitive “pixels,” each of which is an individual light receptor. Above the pixel layer 302 is a filter layer 304 comprising a pattern of red (“R”), green (“G”) and blue (“B”) filters (the pattern shown is commonly called a “Bayer” filter). The filter layer 304 is shown spaced from the pixel layer 302 for clarity, but typically there is little or no gap between the layers and each individual pixel can only receive light that passes through one filter. Each filter has a peak transmission value at one of the three primary colors, and each filter allows the underlying pixel to receive only a selected range of wavelengths surrounding the particular primary color of the filter. In this type of sensor, the locations of the filter colors will not always coincide with the locations of the light having a wavelength that will pass through the filter, which can lead to some sampling errors. For example, a very small (“pinpoint”) colored light source may directly strike a pixel covered by a different-colored filter, and only partially strike a filter of the same color, which can lead to an erroneously small intensity value measurement for the light source. Furthermore, because the filters are physically offset from one another, it is necessary to interpolate the physical locations and intensities of the data obtained from the pixels receiving red, green and blue light, in order to generate a “full-color” image that represents the physical locations and intensities of the light sources in the image. So-called demosaicing and de-Bayering algorithms are commonly used for this purpose, as known in the art. While such algorithms are considered to be very good at reconstructing the original image's feature locations, they are not able to provide a perfect reconstruction of the original image.

Where the color differentiation between the nucleobase labels is significant, a conventional color digital sensor may be used to simultaneously read all of the nucleobase labels present in the field of view of the sequencing surface 102. An exemplary process would include the following steps: first, extend the DNA templates in the presence of all four labeled nucleobases to add one of the four nucleobase labels to each DNA template; second, excite the sequencing surface 102 with all four light sources 204; third, operate the camera 212 to capture an image of the sequencing surface 102 showing the emitted light from all four nucleobase labels; fourth, process the image data to determine which nucleobase label has bonded with each DNA template; and then repeat the foregoing steps. If the sequencing surface 102 is larger than the field of view of the objective lens 106, the steps of exciting and capturing may be repeated at multiple locations along the sequencing surface 102 by moving the objective lens 106 or the sequencing surface 102. Alternatively, the sequencing surface 102 may be scanned by capturing a time-dependent sequence of images as the sequencing surface 102 is moved using the movable stage 118 or by traversing the optics over the surface 102. Other steps used in typical SBS instruments are omitted for clarity, but can be included in the process as would be appreciated by a person of ordinary skill in the art.

It is expected that in some cases the conventional color digital sensor will not be able to accurately differentiate between different wavelengths emitted by particular nucleobase labels. One reason for this may be that the red, green and blue filters in conventional color digital cameras typically have broad spectral ranges with significant amounts of overlap in their spectral ranges (for example, the “red,” “green” and “blue” filters all may transmit some light in the middle green range at about 540 nm). This leads to cross-talk among the color values and yields uncertainty in the final color determination. Thus, a conventional color sensor may not be able to differentiate with the desired accuracy between certain emission wavelengths in the yellow and green spectra. In such cases, the above process may be modified by selectively activating each of the first, second, third and fourth light sources 204 in sequence, and operating the camera 212 to capture an image of the sequencing surface 102 once during each of the four light source activation cycles. Using this technique, all four nucleobase labels can be rapidly read, without requiring any movement of the parts. Alternatively, if it is found that the conventional color sensor can differentiate between some emission wavelengths, but not others, the light sources 204 may be activated in groups that do not present differentiation problems (e.g., activate “blue,” “yellow” and “red” in a first cycle, and “green” in a second cycle, or activate “blue” and “yellow” in a first cycle, and “green” and “red” in a second cycle), and the camera 212 may be operated to read the nucleobase labels once per activation cycle to read two types of nucleobase labels at a time. Furthermore, if the light sources 204 are operated in groups, then an embodiment also may use multiple suitable two-pass dichroic mirrors 208 that are selectively moved into the optical path during each light activation cycle. Other alternatives will be apparent to persons of ordinary skill in the art in view of the present disclosure.

The camera 212 alternatively may comprise a hyperspectral camera that is configured to directly detect the emission wavelengths of the nucleobase labels being used in the instrument, and preferably only those emission wavelengths. Unlike conventional color cameras, hyperspectral cameras are able to directly detect particular wavelengths without having to interpolate color information that has passed through red, green and blue filters. For example, as shown in FIG. 4, a hyperspectral camera sensor 400 may use a generally conventional sensor layer 402, but replace the conventional filter layer 304 with a filter array 404 tuned to pass the wavelengths λ₁, λ₂, λ₃, λ₄ emitted by the nucleobase labels to separate pixels on the sensor layer 402. Each wavelength λ₁, λ₂, λ₃, λ₄ may be selected to correspond to a peak value of emission light for each nucleobase label, but it will be appreciated that other values may be selected. In one example, these wavelengths include a first range including about 525 nm, a second range including about 565 nm, a third range including about 630 nm, and a fourth range including about 680 nm, but other embodiments may use different wavelengths.

It will also be appreciated that each wavelength λ₁, λ₂, λ₃, λ₄ may comprise a range of wavelengths. For example, each wavelength λ₁, λ₂, λ₃, λ₄ may comprise a peak emission value for one of the nucleobase labels, plus a range not exceeding about 20 nm surrounding the peak value. This is expected to provide greater differentiation of the different nucleobase labels without unduly reducing the light intensity. If greater differentiation is desired, the range surrounding the peak value may be reduced to a range not exceeding about 5 nm, but the signal to noise ratio may be reduced in this embodiment. It is also envisioned that one or more of the wavelengths λ₁, λ₂, λ₃, λ₄ may comprise a range of wavelengths that does not include the peak emission wavelength for a particular nucleobase label. This may be helpful where the peak emission wavelength of a first nucleobase label is close to a significant emission intensity of a second nucleobase label, but the first nucleobase label emission range otherwise includes a relatively intense and readable region that is more distinct from the second nucleobase label emission range. Other alternatives will be apparent to persons of ordinary skill in the art in view of the present disclosure. It is also preferred, but not strictly required, that the wavelengths λ₁, λ₂, λ₃, λ₄ do not comprise overlapping wavelengths. As used herein, “overlapping wavelengths” includes overlap of significant amounts of light intensity at any particular wavelength (e.g., overlap within the full width at half maximum portion of the transmitted range of wavelengths). Some insignificant overlap may occur due to mirrors or filters not providing 100% efficiency at reflecting or blocking wavelengths outside the desired range, but where such inefficiencies do not yield appreciable changes to the analyzed image data, such inefficiencies would not be considered to result in “overlapping wavelengths.”

The hyperspectral filter array 404 may comprise, for example, a number of Fabry-Perot spectral filters that each transmit only a narrow range of wavelengths (e.g., 5-20 nm FWHM (full-width half maximum)). An example of a Fabry-Perot filter is shown in FIG. 5. In this example, the filter 500 comprises parallel first and second mirrored surfaces 502, 504 that are separated by a distance L to form a gap 506. Light passes through one of the surfaces 502 and enters the gap 506. Inside the gap 506, multiple interference causes the filter output spectral characteristic to peak sharply over a narrow band of wavelengths. The transmitted wavelength depends on the angle of incidence θ, and the distance L between the surfaces 502, 504, according to known equations. The range of wavelengths transmitted through the Fabry-Perot spectral filter can be tuned by adjusting the reflectivity of the surfaces 502, 504, with more reflective surfaces yielding a narrow transmission band (so-called higher “finesse”). Such Fabry-Perot filters and other suitable devices are known in the art, and need no further explanation here. Suitable hyperspectral filters are available from IMEC International of Heverlee, Belgium. In other embodiments the hyperspectral filter array 404 may be configured to detect an even greater number of wavelengths.

The hyperspectral sensor has the advantage that it does not need to interpolate red, green and blue data to determine the wavelengths of the light sources generated by the image, which improves the color accuracy and can reduce the processing power required to interpret the input signal. In sequencing systems with nucleobase labels having relatively closely-spaced emission wavelengths, it is expected that a hyperspectral sensor with sensor pixels tuned to the emission wavelengths will be able to differentiate between the emission wavelengths and provide a suitable output for accurately determining which nucleobase labels have bonded with each DNA template. The separate detection of the individual emission wavelengths also provides the possibility to use the spectral information between the color “channels” for spectral cross talk analyses, such as an analysis to determine the influence of each individual excitation beam wavelength on the intensities of all of the different nucleobase labels. This kind of analysis can be used to establish cross-talk parameters and relationships, and to recalculate emission signal intensities in real time. Furthermore, a hyperspectral camera can be tailored to read nucleobase labels that emit at virtually any wavelength, whether the wavelength is visible to the human eye or not.

In the example of FIG. 4, the hyperspectral filter array 404 uses a mosaic pattern of filters. Thus, it may be necessary to perform a demosaicing algorithm on the raw data to more accurately determine the locations of the detected light sources.

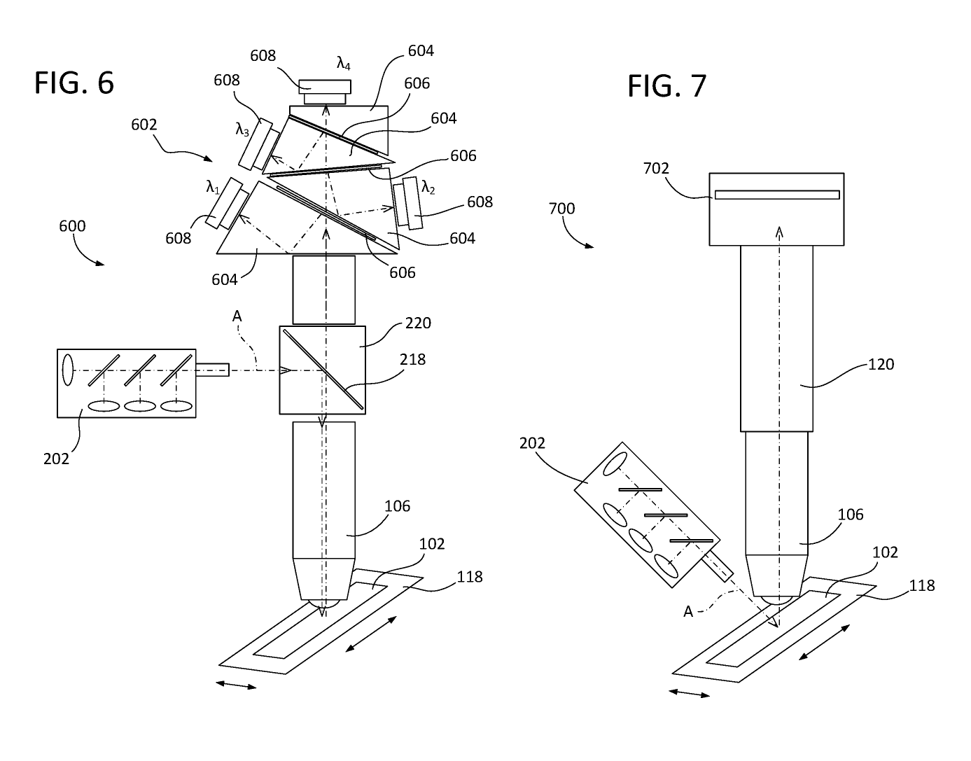

A further embodiment, shown in FIG. 6, is generally the same as the embodiment of FIG. 2, but the instrument 600 uses a multiple-sensor camera 602 rather than a single-sensor camera 212. The illustrated multiple-sensor camera 602 is a hyperspectral camera having an arrangement of prisms 604 that separate the emitted light into the four different wavelengths λ₁, λ₂, λ₃, λ₄ that are emitted by the nucleobase labels used in the sequencing process. Each prism 604 may include a dichroic reflector 606 that reflects light having one of the four wavelengths λ₁, λ₂, λ₃, λ₄ towards a respective sensor 608 to read the color information separately. As explained before, the wavelengths λ₁, λ₂, λ₃, λ₄ each may comprise a peak emission wavelength of a respective one of the nucleobase labels, or may be a range of wavelengths (e.g., not more than about 20 nm, or not more than about 5 nm), and the wavelength ranges preferably do not overlap. The dichroic reflectors 606 may comprise notch filter (i.e., a filter that reflects a particular narrow range of wavelengths), low-pass filters, high-pass filters, or combinations thereof. The four sensors 608 can be optically aligned such that each pixel on each sensor 608 correlates to the same pixel on the other sensors 608, so that it is not necessary to remap the nucleobase label locations when comparing images from one sensor 608 to the other. However, even if the sensors 608 are not optically aligned, it is a routine matter to mathematically remap the images. Multiple-sensor cameras that use dichroic prism separators and multiple sensors (including four-sensor cameras) are commercially available from companies such as JAI A/S of Copenhagen, Denmark and Hamamatsu Photonics K.K. of Hamamatsu, Japan.

A multi-sensor camera 602 is expected to provide a number of advantages. For example, every pixel of each sensor 608 detects all of the light that is transmitted to the sensor 608, so it is not necessary to perform any demosaicing process to reconstruct the exact locations of the nucleobase labels. All else being equal, this provides a somewhat higher resolution image and greater geometric accuracy than systems that use a mosaic filter, and can avoid fidelity loss that might happen when pinpoint colored light only (or mostly) strikes a filter that blocks that wavelength. Separate sensors are also expected to be less subject to inter-pixel cross-talk and noise generation around the fringes of illuminated pixels that might occur when nearby pixels are illuminated by colors of different wavelengths. Separate sensors also can be separately calibrated (e.g., gain control, etc.) to account for different light intensities of the respective wavelengths, and can adjust signal intensities in real time. Other features and advantages will be apparent to persons of ordinary skill in the art in view of the present disclosure.

The use of separate sensors 608 also allows for relatively straightforward calibration and correction of wavelength-dependent phenomena, such as chromatic aberration. Chromatic aberration is caused when a lens does not focus light of different wavelengths at precisely the same point. In a full-color image, this typically manifests as fringes of color towards the outer perimeter of the image frame, where the light is bent to a larger degree by the lenses. At the scale of typical SBS operations, chromatic aberration can be very significant. For example, a nucleobase label emitting in the blue spectrum might appear at the same location as a nearby nucleobase label emitting in the red spectrum, which can lead to false reads. The optical distortion caused by chromatic aberration can be corrected with relative ease when using different sensors for each color. For example, the sensors can be separately focused to eliminate aberration, or the data from each sensor can be separately adjusted using conventional algorithms to reduce or eliminate the aberration before the data is combined to identify the nucleobase label locations.

Other embodiments that use a multiple-sensor camera 602 may separate the component light wavelengths using other devices, such as one or more triangular prisms, or the like. It also is not necessary for the multiple-sensor camera 602 to be a hyperspectral camera. Other embodiments of multiple-sensor cameras 602 may have three sensors to collect red, green and blue wavelengths, and use this data to generate a full-color composite image to read the nucleobase labels. This embodiment could be subject to problems of color differentiation, but such problems can be overcome by sequentially operating the light sources as discussed above in relation to FIG. 2. It will also be appreciated that the ability to simultaneously perform reads on all of the nucleobase labels will depend on whether the dichroic mirror 208 can transmit all four wavelengths at one time. If not, it may be necessary to perform the process at least partly in series and change mirrors 208 between reads, as explained above.

The embodiment of FIG. 2 uses a first optical path to direct the excitation beam to the sequencing surface 102, and a second optical path to direct the emission light to the camera 212. The first and second optical paths both include a shared dichroic mirror 208 that is used to redirect the excitation beam down the objective lens, and in parallel with at least a portion of the emission beam path. In an alternative embodiment, the shared dichroic mirror 208 may be omitted. For example, another embodiment of an instrument is shown in FIG. 7. Instrument 700 includes a conventional sequencing surface 102 and objective lens 106, and may include a light guide 120 or other features to direct the beams. In this embodiment, the combined excitation beam A is transmitted along a first optical path that leads directly to the sequencing surface 102, rather than being reflected by a dichroic mirror to travel parallel to the emitted beam path. Focusing optics, multiband filters and the like (not shown) may be provided along the first optical path. In this embodiment, the first optical path preferably is entirely separate from the second optical path that directs the emitted light to the camera 702. Here, the combined light source 202 is turned (either by turning the source itself or by redirecting the beams using optical elements such as lenses, prisms and mirrors) to direct the excitation beam A obliquely towards the sequencing surface 102, and the objective lens is oriented to read emitted beams traveling perpendicular to the sequencing surface 102. In other embodiments, the excitation beam A may be oriented perpendicular to the sequencing surface 102 and the emitted beam path may be angled obliquely to the sequencing surface 102, or both the excitation beam A and the emitted beam path may be oriented obliquely. Other off-axis arrangements and alternatives will be understood by persons of ordinary skill in the art in view of the present disclosure.

Instrument 700 also includes a camera 702, which may be a conventional color digital sensor camera, a hyperspectral sensor camera, a conventional multi-sensor camera, or a hyperspectral multi-sensor camera. Instrument 700 may be operated like those described previously herein, but removing the dichroic mirror is expected to reduce costs and simplify the instrument design. If desired, one or more excitation filters, emission filters, or other optical components also may be provided in the light paths from the combined source 202 to the sequencing surface 102, and from the sequencing surface 102 to the camera 702. Other alternatives will be apparent to persons of ordinary skill in the art in view of the present disclosure.

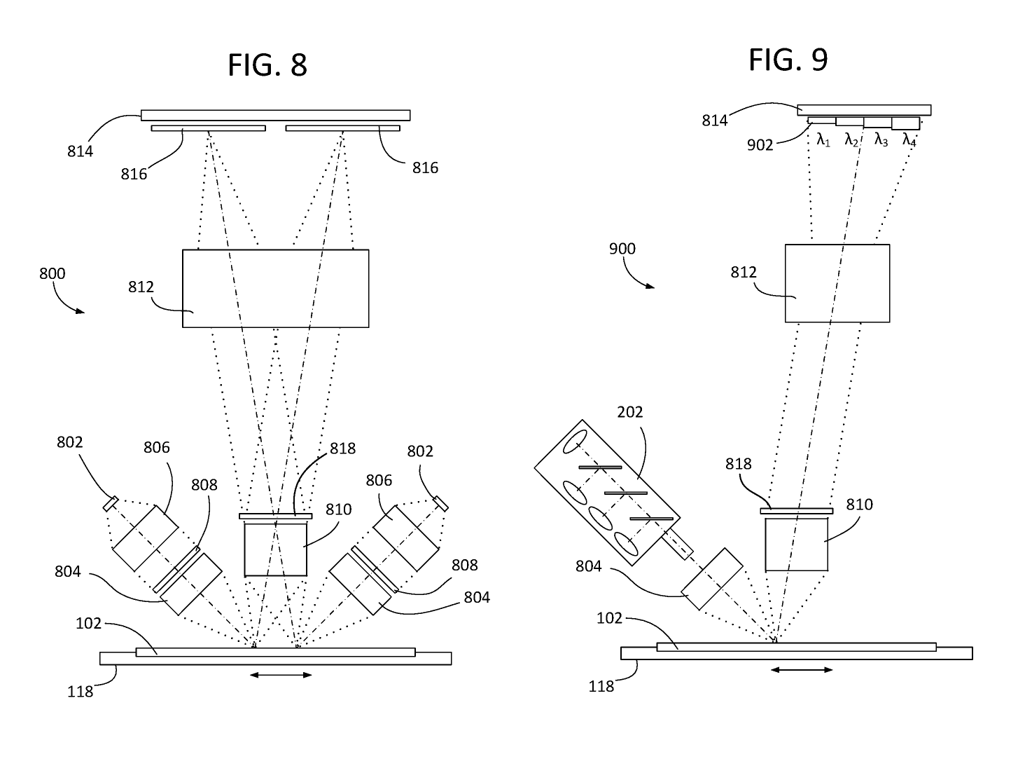

A further example of an off-axis instrument is illustrated in FIG. 8. Instrument 800 is configured to simultaneously read two different nucleobase label colors during continuous scanning of a moving sequencing surface 102. The sequencing surface 102 is mounted on a movable stage 118, but alternatively the sequencing surface 102 may be stationary and the optical system components moved. The instrument 800 has two light sources 802 that are directed towards the sequencing surface 102 through line shape optics 804 (e.g., a cylindrical lens) that bend the excitation light to form a line extending perpendicular to the travel direction and entirely or partially across the width of the sequencing surface 102. A condensing lens 806, excitation filter 808 or other optical components also may be provided in the beam path between the light source 802 and the sequencing surface 102, if desired or necessary.

Emitted light from the sequencing surface 102 is focused by an objective lens 810 towards a projection lens 812, and then to a camera sensor 814 (e.g., a CCD or CMOS sensor). Additional optical features, such as emission filters 818 and beam focusing or shaping lenses, also may be included in the optical path from the sequencing surface 102 to the camera sensor 814. Two emission beam filters 816 are provided between the projection lens 812 and the sensor 814. Each emission beam filter 816 is selected to transmit emission light generated by the activation of one of the light sources 802. For example, the light source 802 on the left might emit light at a first excitation wavelength that causes a first nucleobase label to emit light at a first emission wavelength, and the light source 802 on the right might emit light at a second excitation wavelength that causes a second nucleobase label to emit light at a second emission wavelength that is different from the first emission wavelength.

In use, each light source 802 projects a line-shaped beam onto the sequencing surface 102 at a separate location along the sequencing surface 102, to excite the nucleobase labels at that location. The objective lens 810 and projecting lens 812 transmit light emitted by the nucleobase labels to the sensor 814 via the emission filters 816. The emission beam filter 816 on the right is configured to pass the first emission wavelength to a first part of the sensor 814, and the emission beam filter 816 on the left is configured to pass the second emission wavelength to a second part of the sensor 814. The lenses 810, 812 are configured such that the emitted light generates separate line-shaped beams that strike the first and second parts of the sequencing surface 102. This arrangement of separated excitation beams and separated emission beams provides several advantages. For example, it helps prevent erroneous reads that might occur if an excitation beam excites more than one of the four different nucleobase labels. It also helps isolate the sensor images to help prevent sensor noise and related issues. It will be appreciated, however, that it is not strictly required in all embodiments to separate the locations of the excitation beams.

As the sequencing surface 102 is moved relative to the objective lens 810, the sensor 814 continuously scans across the full or partial width of the sequencing surface 102 to generate a series of images. This time-dependent set of images can be readily collated together into a two-dimensional map of the locations of the nucleobase labels, using algorithms known in the art of line scanning. The sensor 814 simultaneously reads these two-dimensional images for two different nucleobase labels, with each label's emission wavelength being detected at a different location on the sensor 814.

The embodiment of FIG. 8 can be modified to read all four nucleobase label wavelengths. For example, each light source 802 may be changed to emit two excitation wavelengths, one light source 802 may be provided to emit all four wavelengths, or one light source 802 may provide three excitation wavelengths and the other light source may provide one excitation wavelength. In these embodiments, the sensor may be replaced by a conventional color sensor or a hyperspectral sensor such as described above. As another example, two more sets of light sources may be provided to project separate excitation beams at two more different locations on the sequencing surface 102, and emitted light may be read at two more different locations on the sensor 814 after passing through appropriate emission filters.

The embodiment of FIG. 8 also may be modified to use a hyperspectral or regular color sensor. The color sensor may be configured as a mosaic sensor (see, e.g., FIGS. 3 and 4), or as a scanning sensor. For example, FIG. 9 shows a scanning instrument 900 using a hyperspectral filter 902 arranged as a scanning sensor. In this example, a combined light source 202 projects four excitation wavelengths onto the sequencing surface 102 through a line shape optical lens 804. The nucleobase labels emit emission light through an objective lens 810, emission filter 818, and projecting lens 812 to the sensor 814. A scanning hyperspectral filter 902 is located adjacent the sensor 814. The scanning hyperspectral filter 902 is similar to the filter described in relation to FIG. 4, but instead of arranging the different Fabry-Perot spectral filters in mosaic pattern, they are arranged in four rows that extend perpendicular to the movable stage 118 scanning direction. As the sequencing ship 102 is scanned, each row of the scanning hyperspectral filter 902 continuously transmits one of the four nucleobase emission wavelengths to the adjacent sensor pixels, to generate a time-dependent set of images of the locations of nucleobase labels emitting the respective wavelengths. The time-dependent set of images for each wavelength can then be collated into a two-dimensional map for each type of nucleobase label, using algorithms known in the art of line scanning. In this example, the projecting lens 812 may comprise a line shape (e.g., cylindrical) lens that defocuses the emitted light beams to distribute them across the four rows of spectral filters. Other optics and arrangements will be readily appreciated by persons of ordinary skill in the art in view of the present disclosure.

The exemplary embodiments provided and discussed in relation to FIGS. 8 and 9 are expected to provide an advantage over conventional color sensors and hyperspectral sensors that use mosaic patterned filters, because it is not necessary to demosaic the resulting images. These embodiments also may provide an advantage over multi-sensor camera systems because the image data can be collected without using dichroic prisms and multiple sensors to separate and read the different wavelengths (although such devices still could be used in the embodiments of FIGS. 8 and 9). However, it may be necessary to provide more robust and active focusing controls to ensure that the nucleobase labels remain in focus throughout the scanning operation. It also may be more mechanically complex and computationally involved to align the scanned images generated after successive extension processes. These and other considerations will be appreciated by persons of ordinary skill in the art in view of the present disclosure.

The present disclosure describes a number of new, useful and nonobvious features and/or combinations of features that may be used alone or together. It is expected that embodiments may be particularly helpful to increase processing speed in the context of high-throughput nucleic acid sequencing systems, but other benefits may be provided and it will be appreciated that increased processing speed is not necessarily required in all embodiments. While the embodiments described herein have generally been explained in the context of sequencing by syntheses processes, it will be appreciated that embodiments may be configured for use in other sequencing processes that use visual observation of chemical labels. The embodiments described herein are all exemplary, and are not intended to limit the scope of the inventions. It will be appreciated that the inventions described herein can be modified and adapted in various and equivalent ways, and all such modifications and adaptations are intended to be included in the scope of this disclosure and the appended claims.

We claim:

1. A sequencing instrument optical system comprising:
a combined light source comprising a plurality of collinear excitation beams, each excitation beam having a different respective excitation wavelength;
a sequencing surface comprising a plurality of DNA templates and a plurality of nucleobase labels configured to emit a respective emission light at a different respective emission wavelength upon excitation by one or more of the excitation beams;
a color camera configured to detect the emission light of each of the nucleobase labels;
a first optical pathway configured to direct the collinear excitation beams from the combined light source to the sequencing surface; and
a second optical pathway configured to direct the emission light from the sequencing surface to the color camera.

2. The sequencing instrument optical system of claim 1, wherein the combined light source comprises four collinear excitation beams.

3. The sequencing instrument optical system of claim 1, wherein the color camera comprises:
a sensor having a plurality of photosensitive pixels; and
a filter array having a plurality of color filters, each color filter being associated with a respective photosensitive pixel.

4. The sequencing instrument optical system of claim 3, wherein the plurality of color filters comprises red color filters, green color filters, and blue color filters.

5. The sequencing instrument optical system of claim 3, wherein the filter array comprises a hyperspectral filter.

6. The sequencing instrument optical system of claim 5, wherein the hyperspectral filter comprises a plurality of Fabry-Perot spectral filters.

7. The sequencing instrument optical system of claim 5, wherein the hyperspectral filter comprises:
a first group of filters configured to transmit light having a first wavelength associated with a first nucleobase label emission light;
a second group of filters configured to transmit light having a second wavelength associated with a second nucleobase label emission light;
a third group of filters configured to transmit light having a third wavelength associated with a third nucleobase label emission light; and
a fourth group of filters configured to transmit light having a fourth wavelength associated with a fourth nucleobase label emission light.

8. The sequencing instrument optical system of claim 7, wherein:
the first wavelength associated with the first nucleobase label emission light comprises a wavelength corresponding to a first peak emission wavelength of the first nucleobase label;
the second wavelength associated with the second nucleobase label emission light comprises a wavelength corresponding to a second peak emission wavelength of the second nucleobase label;
the third wavelength associated with the third nucleobase label emission light comprises a wavelength corresponding to a third peak emission wavelength of the third nucleobase label; and
the fourth wavelength associated with the fourth nucleobase label emission light comprises a wavelength corresponding to a fourth peak emission wavelength of the fourth nucleobase label.

9. The sequencing instrument optical system of claim 8, wherein the first peak emission wavelength is about 525 nm, the second peak emission wavelength is about 565 nm, the third peak emission wavelength is about 630 nm, and the fourth peak emission wavelength is about 680 nm.

10. The sequencing instrument optical system of claim 7, wherein:
the first wavelength comprises a first distribution of wavelengths having a full width at half maximum value located within a first band of the electromagnetic spectrum;
the second wavelength comprises a second distribution of wavelengths having a full width at half maximum value located within a second band of the electromagnetic spectrum;
the third wavelength comprises a third distribution of wavelengths having a full width at half maximum value located within a third band of the electromagnetic spectrum; and
the fourth wavelength comprises a fourth distribution of wavelengths having a full width at half maximum value located within a fourth band of the electromagnetic spectrum.

11. The sequencing instrument optical system of claim 10, wherein the first band, the second band, the third band and the fourth band do not include any overlapping wavelengths.

12. The sequencing instrument optical system of claim 10, wherein the first band, the second band, the third band and the fourth band each comprises a respective 20 nm wide portion of the electromagnetic spectrum.

13. The sequencing instrument optical system of claim 7, wherein the first group of filters, second group of filters, third group of filters, and fourth group of filters are arranged in a mosaic pattern.

14. The sequencing instrument optical system of claim 7, wherein the first group of filters, second group of filters, third group of filters, and fourth group of filters are arranged in a scanning pattern with each group of filters arranged in a continuous row.

15. The sequencing instrument optical system of claim 14, wherein the sequencing surface is movable in a first direction relative to the color camera, and the first optical path comprises a lens assembly configured to project the collinear excitation beams onto the sequencing surface in a line perpendicular to the first direction.

16. The sequencing instrument optical system of claim 1, wherein the color camera comprises a multi-sensor camera having a plurality of sensors.

17. The sequencing instrument optical system of claim 16, wherein the plurality of sensors comprises three or four sensors, each sensor being configured to receive emission light having a different wavelength.

18. The sequencing instrument optical system of claim 17, wherein the color camera comprises a hyperspectral camera and the plurality of sensors comprises a first sensor configured to detect a first emission wavelength, a second sensor configured to detect a second emission wavelength, a third sensor configured to detect a third emission wavelength, and a fourth sensor configured to detect a fourth emission wavelength.

19. The sequencing instrument optical system of claim 16, wherein the multi-sensor camera comprises a plurality of prisms, each prism being configured to direct a respective emission light to a respective sensor.

20. The sequencing instrument optical system of claim 1, wherein the first optical pathway and the second optical pathway comprises a shared multiband dichroic mirror, the shared multiband dichroic mirror being configured to transmit the emission light, and reflect the plurality of collinear excitation beams towards the sequencing surface.

21. The sequencing instrument optical system of claim 1, wherein at least one of the first optical pathway and the second optical pathway is oblique to the sequencing surface.

22. A sequencing instrument optical system comprising:
a first excitation beam having a first excitation wavelength;
a second excitation beam having a second excitation wavelength that is different from the first excitation wavelength;
a sequencing surface comprising a plurality of DNA templates, a first nucleobase label configured to emit a first emission light at a first emission wavelength upon excitation by the first excitation beam, and a second nucleobase label configured to emit a second emission light at a second emission wavelength upon excitation by the second excitation beam;
a first lens assembly configured to project the first excitation beam onto a first location on the sequencing surface in a line perpendicular to the first direction;
a second lens assembly configured to project the second excitation beam onto a second location on the sequencing surface in a line perpendicular to the first direction, the second location being different from the first location;
a sensor configured to detect the emission light of each of the nucleobase labels, the sensor being movable in a first direction relative to the sequencing surface;
a first color filter configured to transmit the first emission wavelength and located between the first location on the sequencing surface and a first part of the sensor; and
a second color filter configured to transmit the second emission wavelength and located between the second location on the sequencing surface and a second part of the sensor.

23. The sequencing instrument optical system of claim 22, further comprising:
a third excitation beam having a third excitation wavelength;
a third nucleobase label configured to emit a third emission light at a third emission wavelength upon excitation by the third excitation beam;
a third lens assembly configured to project the third excitation beam onto a third location on the sequencing surface in a line perpendicular to the first direction, the third location being different from the first location and the second location; and
a third color filter configured to transmit the third emission wavelength and located between the third location on the sequencing surface and a third part of the sensor.

24. The sequencing instrument optical system of claim 23, further comprising:
a fourth excitation beam having a fourth excitation wavelength;
a fourth nucleobase label configured to emit a fourth emission light at a fourth emission wavelength upon excitation by the fourth excitation beam;
a fourth lens assembly configured to project the fourth excitation beam onto a fourth location on the sequencing surface in a line perpendicular to the first direction, the fourth location being different from the first location, the second location and the third location; and
a fourth color filter configured to transmit the fourth emission wavelength and located between the fourth location on the sequencing surface and a fourth part of the sensor.

25. The sequencing instrument optical system of claim 22, wherein the sequencing surface is mounted on a movable stage to thereby make the sensor movable in a first direction relative to the sequencing surface.

26. The sequencing instrument optical system of claim 22, further comprising one or more lenses configured to project the first emission wavelength along a first discrete line at the first part of the sensor, and to project the second emission wavelength along a second discrete line at the second part of the sensor.
